(19) United States
(12) Patent Application Publication
(10) Pub. No.: US 2013/0196380 A1
(43) Pub. Date: Aug. 1, 2013

(54) IN VITRO PROCESS FOR THE PREPARATION OF ANTIBODIES OF THE IGG TYPE

(71) Applicants: Wolfgang Lüeke, Bad Langensalza (DE); Peter Miethe, Bad Langensalza (DE); Katrin Mueller-Zahm, Bad Langensalza (DE); Ingo Wilke, Bad Langensalza (DE); Gabriele Jansen, Muenster (DE)

(72) Inventors: Wolfgang Lüeke, Bad Langensalza (DE); Peter Miethe, Bad Langensalza (DE); Katrin Mueller-Zahm, Bad Langensalza (DE); Ingo Wilke, Bad Langensalza (DE); Gabriele Jansen, Muenster (DE)

(21) Appl. No.: 13/576,006

(22) PCT Filed: Feb. 4, 2011
(86) PCT No.: PCT/EP2011/051659

§ 371 Date: Jan. 18, 2013

Related U.S. Application Data
(60) Provisional application No. 61/282,875, filed on Apr. 14, 2010.

(30) Foreign Application Priority Data
Feb. 5, 2010 (EP) 10152772.9

(51) Int. Cl. C12P 21/00

(52) U.S. Cl. CPC C12P 21/00
USPC 435/70.3

(57) ABSTRACT

An in vitro process for the preparation of antibodies of the IgG type, comprising the steps of: i. challenging dendritic cells obtained from a donor with an antigen against which the antibodies to be prepared are directed; ii. challenging CD4+ T cells obtained from the same donor with fragments of the antigen presented by the dendritic cells from step L, with the proviso that the dendritic cells are ones essentially not secreting interferon-γ (IFN-γ) and interleukin 12 (IL-12), to generate antigen-specific CD4+ Th2 cells; iii. challenging with the antigen a B cell population obtained from the same donor and including a sufficient proportion of naive B cells; and iv. contacting the antigen-specific CD4+ Th2 cells according to ii. with at least a fraction of the B cell population from iii. to form an antigen-specific plasma cell; v. immortalizing said antigen-specific plasma cell; and vi. isolating the IgG antibodies formed by said antigen-specific plasma cell.

The present invention relates to an in vitro process for the preparation of antibodies of the IgG type.

In modern medicine, the use of diagnostic and therapeutic antibodies has gained a central role. Especially therapeutic antibodies have entered clinical application. Mainly cancers, but meanwhile also neurodegenerative diseases and allergies, are treated with monoclonal antibodies (Nicolaides et al., 2006). The monoclonal antibodies in use today usually represent chimeric (mouse/human) antibodies (human fraction: 70%) or humanized antibodies (human fraction: 95%). In initial human therapy experiments with the original non-humanized monoclonal antibodies almost 20 years ago, it was found that the repeated application led to a strong immune response that severely limited or precluded therapeutic effectiveness (Mascelli et al., 2007).

In order to reach a maximum of safety and tolerability and to eliminate the “residual immunogenicity” of the chimeric or humanized antibodies, methods were developed to allow the preparation of wholly human monoclonal antibodies. These are human phage libraries with the whole diversity of the immunoglobulin genes of human B cells on the one hand, and transgenic mouse systems with the entire human antibody reservoir on the other. A specific immune response against the antigen ovalbumin could be detected with cell populations employed from ovalbumin T cell receptor transgenic mice, but the transfer of these results to an in vitro generation of human antibodies is hardly possible (Heilmann, 2006).

However, all methods for the preparation of the chimeric humanized or human antibodies are very time-consuming and complicated to perform. The immunization of test animals is further necessary (except for the phage libraries), and relatively large amounts of antigen must be provided for this purpose. The consequently necessary heterologous expression systems for recombinant production yield antibodies that do not reliably exhibit the authentic conformation and posttranslational modifications (Konthur et al., 2005). The same applies to antibodies from other species for use in human and veterinary medicine.

In the 1980ies and 1990ies, the first approaches to such in vitro immunization methods were developed. These immunization methods were based on the isolation of the PBMCs, the treatment of the PBMCs with L-leucyl-L-leucine methyl ester (LLME) to remove natural killer (NK) cells and cytotoxic T cells, which suppress the antigen-specific B cell activation, followed by in vitro immunization by the addition of the antigen to the pretreated PBMCs and immortalization with Epstein-Barr virus (EBV; Borrebaeck, 1989). With this method, a number of antibodies, especially antibodies against immunodeficiency virus type 1 (HIV-1), could be prepared (Chin et al., 1995). However, it was found that a sufficient isotype switch from IgM to IgG type could not be achieved with this in vitro immunization method. The antibodies produced are predominantly of the IgM type (Zafiropoulos et al., 1997). Only in recent years, this in vitro immunization method could be improved by the systematic use of the cytokines IL-2, IL-4 and IL-10. The use of these cytokines resulted in the selective induction of Th2 T cells, followed by a clearly enhanced B cell differentiation into antibody-producing plasma cells (Xu et al., 2004). However, antibodies of the IgM type were still preferentially produced. Parallel to this in vitro immunization method for PBMC, a method for the direct in vitro immunization of isolated B cells has also been described recently (Li et al., 2006). In this case too, an enhanced IgM production could only be observed at first.

Bettina König et al report in Immunology, 122, 239-246 about human dendritic cells transfected with allergen-DNA that stimulate specific immunoglobulin G4 but not specific immunoglobulin E production of autologous B cells from atopic individuals in vitro. The aim of this study was to analyse whether DC transfected with allergen-DNA are also able to influence immunoglobulin production of B cells from atopic donors. For this purpose, human monocyte-derived DC from grass-pollen allergic donors were transfected with an adenovirus encoding the allergen Phleum pratense I and cocultured with B cells, autologous CD4⁺ T cells, and CD40 ligand-transfected L-cells. B cells receiving help from CD4⁺ T cells stimulated with allergentransfected dendritic cells produced more allergen-specific IgG4 compared to stimulation with allergen protein pulsed DC or medium, while total IgG4 production was not affected. In contrast, specific IgE production was not enhanced by stimulation with allergen-DNA transfected DC compared to medium and inhibited compared to allergen protein-pulsed DC with similar effects on total IgE production in vitro. Allergen-DNA transfected dendritic cells are able to direct the human allergic immune response from Th2-dominance towards Th1 and Tc1 also resulting in decreased IgE and increased IgG4 production.

WO-A-93/12247 discloses a method of in vitro immunization of cells with an antigen to produce antibodies against that antigen, the method comprising incubating the cells with the antigen and support cells in which the support cells have previously been primed with the antigen prior to incubation with the cells to be immunized.

WO-A-2004/053113 discloses a method of generating lymphocytes specific for particular antigens. More particularly, there is disclosed a method for generating antigen-reactive T-cells and even more particularly cytotoxic (CD8+) T-cells in vitro specific for antigens such as peptide antigens and enables in vitro T-cell priming for particular antigens such as antigens on cancer cells, pathogenic cells, viruses or cells infected with viruses. The method is useful in identifying particularly immunogenic antigens for immunotherapy and further provides the treatment or prophylaxis of a disease or condition in a subject by generating T-cells reactive to an antigenic molecule and administering an effective amount of antigen-reactive T-cells to the subject or other compatible host. The generated dendritic cell/T-cell populations can be used in cellular immunotherapy.

All these examinations relating to the development of an efficient in vitro immunization method indicate that the in-vitro induced immune response is a T cell independent B cell response characterized by insufficient B cell activation, which manifests itself in a highly preferential IgM production and an inefficient isotype switch.

Thus, it is the object of the invention to avoid the drawbacks of the prior art and to replace the complicated methods and approaches of the prior art by simple and reliable preparation methods of antibodies for the IgG type.

This object is achieved by an in vitro process for the preparation of antibodies of the IgG type, comprising the steps of:
i. challenging dendritic cells obtained from a donor with an antigen against which the antibodies to be prepared are directed;
ii. challenging CD4+ T cells obtained from the same donor with fragments of the antigen presented by the dendritic cells from step i., with the proviso that the dendritic cells are ones essentially not secreting interferon-γ (IFN-γ) and interleukin 12 (IL-12), to generate antigen-specific CD4+ Th2 cells;
iii. challenging with the antigen a B cell population obtained from the same donor and including a sufficient proportion of naive B cells; and
iv. contacting the antigen-specific CD4+ Th2 cells according to ii. with at least a fraction of the B cell population from iii. to form an antigen-specific plasma cell;
v. immortalizing said antigen-specific plasma cell; and
vi. isolating the IgG antibodies formed by said antigen-specific plasma cell.

As donors may serve also genetically similar donors such as relatives or donors having the same or close MHC components.

The process steps are performed in the order of their numbering, but it is possible to perform step i. and iii. in parallel or step iii. first or step iii. prior to step ii.

Advantageously, the invention enables the production of authentic antibodies through the induction of a natural immune response directly by human blood cells. This is achieved by the in vitro immunization process of human peripheral blood lymphocytes (PBMC) according to the invention.

Surprisingly, it has been found that an efficient antigen presentation by the dendritic cells is critical to a successful activation of Th2 T cells and thus represents an efficient T helper function for the B cells.

In one embodiment of the inventive process according to claim 1, the dendritic cells are differentiated from peripheral blood monocytes (PBMC) obtained from the donor.

In another embodiment of the inventive process, the antibodies of the IgG type are of the type immunoglobulin G (subtypes 1-4).

In still another embodiment of the inventive process, the donors are mammals including humans.

According to the present invention, in particular, proteins, polypeptides, haptens and immunogenic substances, such as RNA and DNA, can be used as the antigen.

In an embodiment suitable for the preparation of antibodies, the antigen-specific plasma cell may have been immortalized by infection with Epstein-Barr virus (EBV) (Traggiai et al., 2004) or by fusion with a myeloma cell (Köhler & Milstein, 1975).

In still another embodiment, the antigen-specific plasma cell may be cultured in a cell culture or bioreactor, and the antibodies may be recovered from a culture fluid.

The process according to the invention is suitable, in particular, for establishing a monoclonal cell line from an antigen-specific plasma cell.

According to the invention, in particular, a stimulation of the naive B cells and the differentiation thereof into antibody-producing plasma cells with effected isotype switch is caused by the following factors:
1. The binding of an antigen to the B cell receptor is effected.
2. The antigen is subsequently internalized together with CpG (Ruprecht and Lanzavecchia, 2006).
3. Subsequently, an efficient T cell helper function is effected. In particular, Th2 T cells stimulated with the same antigen bind to the MHC class II presented antigen on the B cells by means of their T cell receptor. Such binding can be substantially enhanced by CD40/CD40L interaction.
4. Thereafter, the differentiation into plasma cells and the proliferation thereof can still be enhanced by the cytokine IL-21 (Ettinger et al., 2005).

The inclusion of dendritic cells in the development of an efficient in vitro immunization process as disclosed according to the invention is an essential step for the generation of functional human monoclonal antibodies, of the type IgG.

With the inclusion of monocyte-derived dendritic cells that can ensure an efficient antigen presentation and similarly an efficient T cell activation, it is possible according to the invention to provide a functional in vitro immunization process for the generation of human monoclonal antibodies of the IgG type.

It is to be taken care that the corresponding antigens are guided into the exogenous processing pathway of the dendritic cells with sufficient efficiency, as far as possible, in order to thus ensure the precondition of an optimum and efficient MHC class II antigen presentation. If the natural antigen should escape from the endosomal compartment into the cytosol, which would consequently result in an undesirable cross-presentation of the antigen through MHC class I, the antigen can be “forced” into the exogenous processing pathway by specific surface modifications. This can be realized by the application of well-aimed retargeting strategies with charged polymers, which may be coupled with ligands for specific cellular receptors to ensure the specific uptake into endosomes. To support the processing and final differentiation of the dendritic cells, agonists for endosomal TLR (CpG, RNA) can be co-applied.

The in vitro generation of dendritic cells has long been a great challenge. Experiments for the direct isolation of dendritic cells from donor blood did not prove efficient in the past. This was essentially due to the fact that the yields were very low. This has probably also been the reason why only a T cell independent B cell response was observed in all previous in vitro immunization methods, since an efficient antigen presentation was not possible due to the absence of dendritic cells and consequently no activation of Th2 T cells and thus no T helper function for the B cells was available. Only the development of techniques allowing to generate dendritic cells from defined blood precursor cells led to a fundamental breakthrough. Monocytes that, upon the addition of GM-CSF and IL-4, differentiate into immature DCs capable of efficiently taking up antigens (Peters et al., 1993) could be identified as the essential precursor cells. Further, it could be shown in recent years that the use of IL-15 instead of IL-4 caused the differentiation of monocytes into immature dendritic cells having the characteristics of Langerhans cells (Mohamadzadeh et al., 2001).

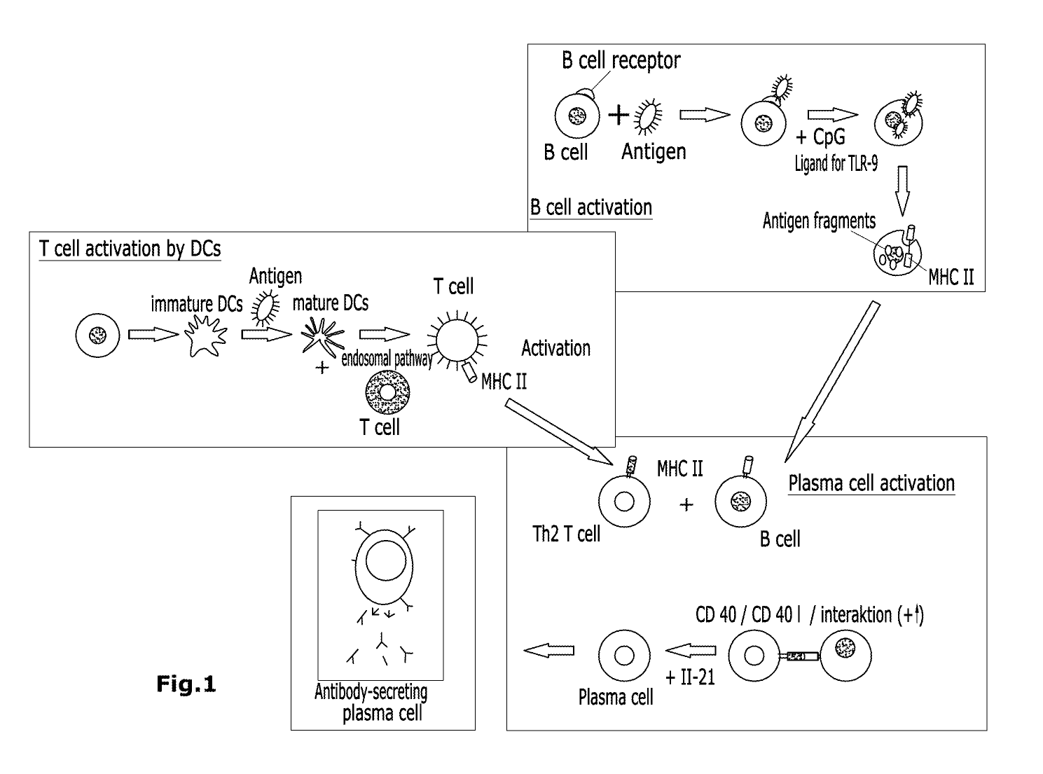

The invention is schematically shown in FIG. 1. The following steps may be pursued:

Generation of the Dendritic Cells

Experiments for the direct isolation of DCs from donor or patient blood proved to be inefficient in the past. Among others, this was due to the fact that the yields were very low. Only the application of techniques allowing to generate DCs from defined blood precursor cells led to an improvement. Monocytes that, upon the addition of additives like GM-CSF and IL-4, differentiate into immature DCs capable of efficiently taking up antigens (Peters et al., 1993) were identified as the precursor cells that can be employed according to the invention. The use of IL-15 instead of or together with IL-4 causes the differentiation of the monocytes into immature dendritic cells having the characteristics of Langerhans cells (Mohamadzadeh et al., 2001). These dendritic cells additionally proved to be potent antigen-presenting cells.

For the generation of the monocyte-derived dendritic cells (MDDC), three methods have been established in the prior art, namely the enrichment of the monocytes by adherence to plastic, the indirect isolation of the monocytes, or the positive selection of the monocytes. According to the invention, the monocyte-derived dendritic cells can be generated especially by indirect isolation of the monocytes.

Antigen Uptake and Processing

The antigen uptake by the dendritic cells, the subsequent processing and presentation affects the resulting immune response. Therefore, it is preferred according to the invention that the antigen is taken up into intracellular endosomal compartments, which leads to the specific proteolytic processing of the protein, and that an efficient antigen presentation through MHC class II molecules thus occurs via this exogenous processing pathway, which is advantageous for the in vitro generation of the antibodies of the IgG type according to the invention.

For an efficient MHC class II presentation, the guiding of the antigen into the exogenous processing pathway can be achieved by coupling the antigen to charged polymers (depending on the corresponding antigen) coupled to ligands for specific cellular receptors. It may be helpful to couple the antigen to substances like, for example, polycations or polyanions. In particular, the antigen is coupled to polyethyleneimine, wherein a ratio of from 1:2 to 1:10, especially 1:5, can be observed, for example.

The Maturation and Antigen Presentation is Optionally Influenced

A satisfactory maturation and antigen presentation of dendritic cells after the uptake of antigen is associated with the efficient induction and secretion of interferons of class-I (IFN-α/β), as was shown using viruses as an example (Kawai and Akira, 2006). This function can be substantially enhanced by the binding of virus-specific ligands to particular toll-like receptors (TLR). In viruses getting into the cell by endocytosis, the TLR 7/8 and 9 play an important role. Evidently, these TLR bind to the released viral genomes in the endosomes. TLR 7/8 seem to be responsible for the binding to single-stranded RNA genomes, while TLR 9 binds to non-methylated CpG motifs in viral DNA genomes (Kawai and Akira, 2006). Therefore, preferably the ligands for TLR 7/8 (ssRNA) and 9 (CpG-ODN) are packaged into virus-like particles (VLP). The VLP are loaded with polymers (preferably polyethyleneimine) identical to those of the co-applied antigen to thereby ensure the uptake into the identical cell compartment.

Polarization of Naive CD4+ T Cells

The generation of CD4+ T helper cells of Th2 type is important to the induction of an adaptive humoral immune response. What is advantageous for this purpose is the MHC class II antigen presentation by dendritic cells and the high regulation of the co-stimulating surface molecules CD40, CD80, CD83, CD86 and OX40, as is typical of matured dendritic cells. Further, the cytokine environment is not to be underestimated. After the binding of the naive CD4+ T cell through the T cell receptor to the antigen-presenting MHC class II molecules on the dendritic cells, the polarization of the bound T cell into Th1 or Th2 cells is initiated. A co-stimulation by the interaction of CD80/CD86 and CD80L/CD86L (CD28) can cause polarization into Th2 cells. OX40/OX40L can be employed for enhancement. Further, the ratio of dendritic cells to naive CD4+ T cells is important to the polarization into Th2 cells (Tanaka et al., 2000). Therefore, according to the invention, especially the antigen-loaded dendritic cells are employed at a ratio to the naive CD4+ T cells of from 1:50 to 1:800, especially 1:300. Further, the cytokines IL-4, IL-5, IL-6 and IL-10 can be used according to the invention.

Activation of Naive B Cells

For an efficient activation of the B cells, the three measures mentioned below are advantageous, in particular: stimulation of the B cell receptor, co-stimulation through CD40/CD40L by antigen-specific Th2 helper cells, and the interaction with TLR 7/9 agonists. In the present invention, for example, the naive B cells are incubated with antigen and TLR 7/9 agonists for an efficient activation. The activation is supported by co-stimulation with anti-CD40 antibodies or antigen-specific Th2 T helper cells.

Orchestration of the Individual Components

For the development of a practical system, the optimized individual components can be assembled to a functional overall system, and the antibodies may optionally be purified and characterized. This process is illustrated in an exemplary manner using a bacterial toxin as an example.

Bacterial toxins belong to the most toxic substances occurring in nature. Due to their high toxicity, a direct immunization using them is not possible. Rather, different toxoids were employed in which the toxicity is usually highly reduced by an aldehyde treatment. These detoxification reactions cannot be controlled practically, so that immunization is performed with relatively random surface epitopes. Corresponding animal-intensive experiments for the generation of both monoclonal and polyclonal antibodies against toxins were published on several occasions. Despite great efforts, only a few antibodies or sera with an acceptable affinity for the native toxin could be produced so far with this approach. Rather, the antibodies usually exhibit a much higher affinity for the toxoid or undergo cross-reactions with other aldehyde-protein adducts. When used in diagnostic test systems, they could achieve only moderate sensitivities and specificities. Therefore, it is of great importance to establish a highly flexible system for the generation of diagnostic and therapeutic antibodies for human application.

The invention is further illustrated by means of the following Examples.

### EXAMPLES

### Example 1

Production of Monoclonal IgG Antibodies Against Botulinum Toxin A

1.1. Generation of Sufficient Amounts of Peripheral Blood Lymphocytes

The starting cell population from which the dendritic cells and the cells serving for the generation of the B cell clones are prepared are peripheral blood lymphocytes (PBMC). For the withdrawal of blood, 9.5 ml monovettes containing K+/EDTA as an anticoagulant were used. Forty ml of whole blood were withdrawn from the donors.

Fifteen ml each of separation medium was covered by a layer of 20 ml of the blood. The density gradient centrifugation was effected at 600×g for 20 min without a brake.

The interphase was withdrawn and subsequently washed (600×g, 10 min, 4° C.). The cells were typed by means of flow cytometry of the expression status of the surface markers CD3, CD4, CD8 and CD20.

The lymphocytes in a cell number of 1-6×10⁷ cells and the monocytes in a cell number of 2-5×10⁶ were cryopreserved in the presence of DMSO and autologous serum and stored on liquid nitrogen.

1.2. Purification and Characterization of the Lymphocyte Subpopulations

CD4+ T cells: The CD4+ T cells are of particular importance to this project. Therefore, it was important to isolate the cells from the entire population of the PBMC by means of a separation kit. The RosetteSep® Human CD4+ T Cell Enrichment Kit from the company StemCell Technologies was used for this purpose.

Subsequently, the cells were counted, cryopreserved in the presence of DMSO and autologous serum and stored on liquid nitrogen. To the following experiments, it is particularly important what Th1/Th2 ratio is observed and whether “memory” CD4+ T cells are present. In order to be able to examine the accessory activity of the dendritic cells (antigen uptake, processing, presentation, lymphocyte activation) in a standardized way, a lymphocyte proliferation assay was performed.

BoNT/A, virus-like particles (VLP, recombinant form of the human polyoma virus JCV), tetanus toxoid as a standard antigen and the polyclonal mitogen phytohemagglutinin (PHA) were used as stimulation antigens.

Thus, monocytes were differentiated into immature dendritic cells (DC) and plated onto a sterile flat-bottomed 96-well plate at 1×10⁴ per well. Five different charges (five stimulation antigens) were performed:

To check the lymphocyte stimulation by botulinum toxin, 2.5 μg of BoNT/A per well was employed. As a positive control, on the one hand, tetanus toxoid was employed at a concentration of 150 Lf/ml. To the DC, 50 μl (7.5 Lf) of tetanus toxoid (TT) was pipetted. On the other hand, 100 μl of PHA parent solution with a concentration of 10 μg/ml (final concentration: 5 μg/ml) was added to the DC. Further, 10 mg of VLP solution per well was added. As a negative control, pure medium was added to the cells.

For antigen uptake, the DC were incubated with the various stimulants at 37° C. for four hours. Autologous lymphocytes at 1×10⁵ per well were added. The lymphocyte proliferation activity was tested by means of a CellTiter GloLuminescent Cell Viability Assay from Promega.

B cells: In the same way as the CD4+ T cells, the B cells were isolated from the entire population of the PBMC by means the separation kit RosetteSep® Human B Cell Enrichment Kit from the company StemCell Technologies.

In the same way as the CD4+ T cells, the B cell population was examined for the ratio of “naive” to “memory” B cells. This was determined by the analysis of defined surface markers by means of flow cytometry (CD19, CD4, CD27, CD5). The expression pattern of the surface markers provided information about the ratio of “naive” to “memory” B cells. In parallel, the serological status of the donors was determined.

Then, only the PBMC from those donors who had a low and defined immune status were used for the further studies.

1.3. Dendritic Cells

A. Generation
1. Enrichment of the monocytes by adherence to plastic.
2. Generation of the DC with 800 U/ml GM-CSF and 500 U/ml IL-4 in the DC medium (CellGenix) for 5 days.
3. Characterization of the immature DC by means of flow cytometry (CD 1a, HLA class I & II molecules, CD 80, CD 83, CD 86).

B. Loading and Maturation
1. Loading of 10 μg of botulinum toxin with polyethyleneimine.
2. Loading of the MDDC with botulinum toxin/PEI complexes.
3. Examination of the differentiation of the loaded MDDC by means of flow cytometry (CD 1a, HLA class I & II molecules, CD 80, CD 83, CD 86).
4. Determination of the IFN-α/β/γ as well as the IL-6, IL-10 and IL-12 secretion after different periods of time.

C. Co-Application of TLR Agonists by Means of Virus-Like Particles (VLP)
1. The nucleic acids (ODN 2395 or R848) are packaged into virus-like particles (VLP).
2. The VLP are loaded with the same polymers (PEI) as the co-applied botulinum toxin to ensure uptake into the same cell compartment.

1.4. Incubation of the Dendritic Cells with Naive CD4+ T Cells

The antigen-loaded dendritic cells are incubated with the naive CD4+ T cells at a ratio of 1:300.

The secretion of the cytokines IL-4, IL-5, IL-6, IL-10, IL-12 and IFN-γ is followed over time. Further, the expression of the Th2 surface markers CCR2, 3 and 4 is examined. The generation of Th2 T cells is also performed in the presence of antibodies against IL-12 and IFN-γ (cytokines favoring Th1).

1.5. Activation of Naive B Cells

Thus, the naive B cells were isolated in a highly pure form from the PBMC. To determine the antigen-specific activation, the B cells are incubated with the various forms of the botulinum toxin together with TLR7/9 agonists (ODN 2395 or R848). The activation is supported by the costimulation with anti-CD40 antibodies or antigen-specific Th2 T helper cells.

1.6. Orchestration of the Individual Components

Antibody-Secreting Plasma Cells
1. Generation of 1×10⁶ dendritic cells for 5 days with 500 U/ml IL-4 and 800 U/ml GM-CSF.
2. Addition of 10 μg of BoNT/A to the immature dendritic cells.
3. Addition of CD4+ T cells to mature dendritic cells at a ratio of 300 to 1 for the generation of Th2 T helper cells.
4. For the antigen-specific activation, the B cells were incubated with the botulinum toxin together with TLR7/9 agonists. The activation is supported by the addition of the cytokines IL-2, IL-4, IL-6, IL-10 and IL-21.
5. Mixing of the activated B cells with the activated Th2 T helper cells.
6. Incubation for up to 10 days, the antibody secretion being checked each day.

1.7. Generation of B Cell Clones

Isolation and Immortalization of the B Cells

To recover the B cell clones, the activated B cells are isolated from the corresponding system and immortalized with a new method (Traggiai et al., 2004). Thus, the isolated B cells are plated in microtitration plates at a low cell number, restimulated with the corresponding botulinum toxin and the corresponding TLR agonist, and immortalized with Epstein-Barr virus.

The B lymphocytes were sown at a cell number of 10 or 50 cells per 96 U-bottom well with 50,000 mononuclear cells in medium with 2.5 μg/ml CpG 2006 and 30% supernatant of B95-8 cells. After about two weeks, the supernatants of the cultures were examined for antibody production.

1.8. Characterization of the Antibodies

A. Purification of the Antibodies

About two weeks after the restimulation and immortalization, the supernatants of the cultures are examined for antibody production, and the positive cultures are biologically cloned and expanded. The secreted antibodies are purified from the supernatants by means of affinity chromatography (Amersham).

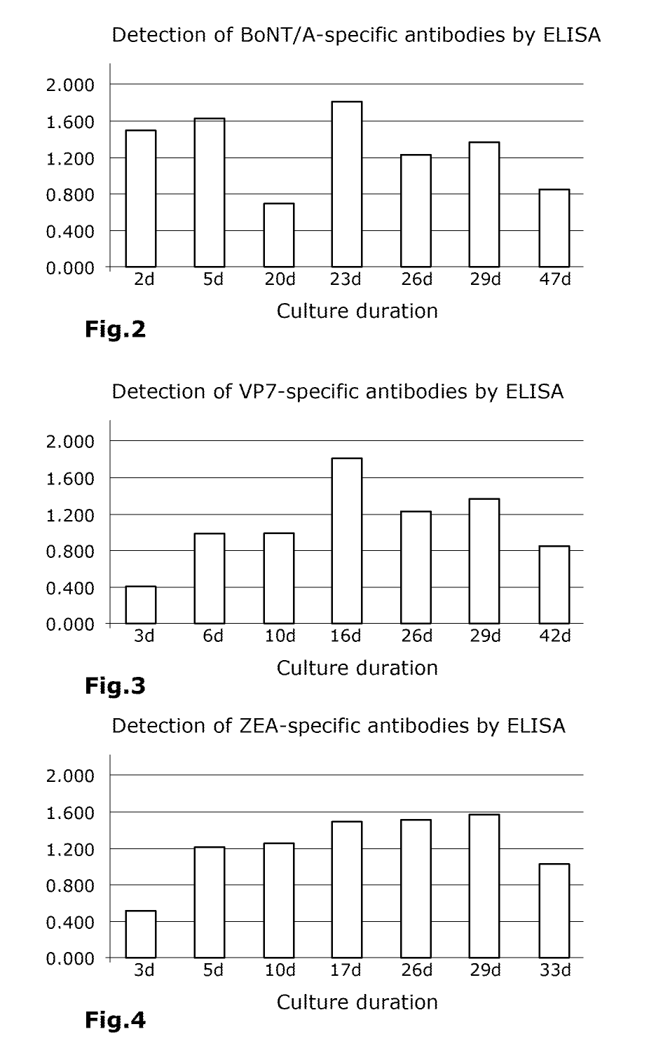

FIG. 2: Use of the in-vitro generated AB as a coating antibody in sandwich ELISA

B. Neutralization Capacity

For neutralization examinations, the binding inhibition of the botulinum toxin to various target cells was determined by means of the purified antibodies. Thus, the antigen was incubated with different antibody concentrations (diluted in 2-log steps) at RT for 45 min. This charge was subsequently added to 1.5×10⁵ target cells/ml and incubated at 37° C. for 3 days. The detection of the bound botulinum toxin was then effected in Western blot. The antibody concentration (ng/ml) at which a complete protection was found in the cell cultures was determined as the neutralizing antibody concentration.

### Example 2

Production of Monoclonal IgG Antibodies Against African Horse Sickness (VP7)

2.1. Generation of Sufficient Quantities of Peripheral Blood Lymphocytes

9.5 ml monovettes containing K+/EDTA anticoagulant were used for blood collection. Four hundred milliliters of whole blood was withdrawn from the horses. Twenty milliliters of the blood was layered over 15 ml each of separation medium. Density gradient centrifugation was performed at 600 g for 20 min with brake switched off. The interphase was removed and subsequently washed (600 g, 10 min, 4° C.). The cells were typed for the expression state of surface markers CD3, CD4, CD8, CD19 and CD45 by flow cytometry. Cryopreservation was effected at a cell count of 6×10⁷ cells for the lymphocytes and 5×10⁶ for the monocytes in the presence of DMSO and autologous serum, followed by storage at −150° C.

2.2. Purification and Characterization of the Lymphocyte Subpopulations

CD4+ T cells: The CD4+ T cells were isolated from the total PBMC population by means of a separation kit. For this purpose, the RosetteSep® Human CD4+ T Cell Enrichment Kit from the company StemCellTechnologies was used. The cells were subsequently counted and cryopreserved in the presence of DMSO and autologous serum, and stored at −150° C.

B cells: In a similar way as with the CD4+ T cells, the B cells were isolated from the total PBMC population by means of the separation kit RosetteSep® Human B Cell Enrichment Kit from the company StemCellTechnologies.

In a similar way as with the CD4+ T cells, the B cell population was examined for the ratio of “naive” to “memory” B cells. The latter was determined by analyzing defined surface markers by flow cytometry (CD19, CD20, CD21, CD27). The expression pattern of the surface markers revealed the ratio of “naive” to “memory” B cells. Then, for the further studies, only the PBMC from those donors who have a low and defined immune status were used.

2.3. Dendritic Cells (DCs)

A. Generation

1. Enrichment of monocytes by adherence to plastic.

2. Generation of DCs with 1000 U/ml of equine GM-CSF and 30 ng of equine IL-4 in DC medium (CellGenix) for 6 days.

3. Characterization of immature DCs by flow cytometry (CD1a, HLA class I & II molecules, CD80, CD83, CD86).

B. Loading and Maturing

1. Loading of the DCs with 5 μg of VP7.

2. Examination of the differentiation of the loaded DCs by flow cytometry (CD1a, HLA class I & II molecules, CD80, CD83, CD86).

3. Assay of the IFNα/β/γ as well as IL-6, IL-10 and IL-12 secretion after different periods of time.

C. Co-Application of TLR Agonists using Virus-Like Particles (VLPs)

1. The nucleic acids (ODN 2395 or R848) are packaged into virus-like particles (VLPs).

2. The VLPs are loaded with polymers (PEI) to ensure uptake.

2.4. Incubation of the Dendritic Cells with Naive CD4+ T Cells

The antigen-loaded dendritic cells were incubated with the naive CD4+ T cells at a ratio of 1:70.

The secretion of cytokines IL-4, IL-6, IL-10, IL-12 and IFN-γ is monitored over time. Further, the formation of Th2 surface markers CCR2, 3 and 4 is examined.

2.5. Activation of Naive B Cells

Thus, the naive B cells were isolated from the PBMCs in a highly pure form. In order to determine the antigen-specific activation, the B cells with the VP7 are incubated together with TLR7/9 agonists (ODN 2395 or R848). The activation is supported by antigen-specific Th2-T helper cells.

2.6. Orchestration of the Individual Components

Antibody-Secreting Plasma Cells

1. Generation of 1×10⁶ dendritic cells for 6 days with 30 ng of equine IL-4 and 1000 U/ml of equine GM-CSF.

2. Addition of 5 μg of VP7 to the immature dendritic cells.

3. Addition of CD4+ T cells to mature dendritic cells at a ratio of 70 to 1 to generate Th2-T helper cells.

4. For antigen-specific activation, the B cells were incubated with the VP7. The activation is supported by the addition of cytokines IL-2, IL-4, IL-6, IL-10.

5. Admixing the activated B cells with the activated Th2-T helper cells.

6. Incubation for up to ten days, the antibody secretion being checked each day.

2.7. Generation of B Cell Clones

Isolation and Immortalization of B Cells

In order to recover the B cell clones, the activated B cells are isolated from the corresponding system, and hybridomas may be formed by fusion with a myeloma cell. Selection generates an immortal cell line, which can be cultured in vitro and produces antibodies of a single specificity and isotype (Köhler & Milstein 1975). For this purpose, the lymphocytes were isolated from the system using a B-Cell Isolation Kit (Miltenyi Biotec). Subsequently, the cells were fused with the mouse myeloma cell line SP2/0 in the presence of polyethylene glycol (PEG). The cell seeds were sown at a low cell count in DMEM (20% fetal bovine serum, 1×10⁻⁴ M hypoxanthine, 4×10⁻⁴ M aminopterine and 6×10⁻⁵ M thymidine) in 96-well plates. After about two weeks, the supernatants of the cultures were examined for antibody production (Perryman L E et al., 1990).

2.8. Characterization of the Antibodies

A. Purification of the Antibodies

About two weeks after the fusion, the supernatants of the cultures were examined for antibody production, and the positive cultures were biologically cloned and expanded. The secreted antibodies were purified from the supernatants by affinity chromatography (Amersham).

B. Neutralization Capacity

For neutralization studies, the inhibition of the binding of the VP7 protein to different target cells was determined by means of the purified antibodies. Thus, the antigen was incubated with different antibody concentrations (diluted in 2-log steps) at RT for 45 min. This mixture was subsequently added to 1.5×10⁵ target cells/ml and incubated at 37° C. for 3 days. The detection of the bound VP7 was then effected by Western blotting. A concentration (ng/ml) at which complete protection was found in the cell cultures was shown as the neutralizing antibody concentration. FIG. 3 shows detection of in vitro generated VP7-specific antibody by ELISA.

### Example 3

Production of Monoclonal IgG Antibodies Against the Mycotoxin Zearalenone (ZEA)

3.1. Generation of Sufficient Quantities of Peripheral Blood Lymphocytes

9.5 ml monovettes containing K+/EDTA anticoagulant were used for blood collection. Forty milliliters of whole blood was withdrawn from the donors. Twenty milliliters of the blood was layered over 15 ml each of separation medium. Density gradient centrifugation was performed at 600 g for 20 min with brake switched off. The interphase was removed and subsequently washed (600 g, 10 min, 4° C.). The cells were typed for the expression state of surface markers CD3, CD4, CD8, CD19 and CD45 by flow cytometry. Cryopreservation was effected at a cell count of 1-6×10⁷ cells for the lymphocytes and 2-5×10⁶ for the monocytes in the presence of DMSO and autologous serum, followed by storage at −150° C.

3.2. Purification and Characterization of the Lymphocyte Subpopulations

CD4+ T cells: The CD4+ T cells were isolated from the total PBMC population by means of a separation kit. For this purpose, the RosetteSep® Human CD4+ T Cell Enrichment Kit from the company StemCellTechnologies was used. The cells were subsequently counted and cryopreserved in the presence of DMSO and autologous serum, and stored at −150° C.

B cells: In a similar way as with the CD4+ T cells, the B cells were isolated from the total PBMC population by means of the separation kit RosetteSep® Human B Cell Enrichment Kit from the company StemCellTechnologies.

In a similar way as with the CD4+ T cells, the B cell population was examined for the ratio of “naive” to “memory” B cells. The latter was determined by analyzing defined surface markers by flow cytometry (CD19, CD20, CD21, CD27). The expression pattern of the surface markers revealed the ratio of “naive” to “memory” B cells. Then, for the further studies, only the PBMC from those donors who have a low and defined immune status were used.

3.3. Dendritic Cells (DCs)

A. Generation

1. Enrichment of monocytes by adherence to plastic.

2. Generation of DCs with 800 U/ml of GM-CSF and 500 U/ml of IL-4 in DC medium (CellGenix) for 5 days.

3. Characterization of immature DCs by flow cytometry (CD1a, HLA class I & II molecules, CD80, CD83, CD86).

B. Loading and Maturing

1. Loading of the DCs with 5 μg of ZEA.

2. Examination of the differentiation of the loaded DCs by flow cytometry (CD1a, HLA class I & II molecules, CD80, CD83, CD86).

3. Assay of the IFNα/β/γ as well as IL-6, IL-10 and IL-12 secretion after different periods of time.

C. Co-Application of TLR Agonists using Virus-Like particles (VLPs)

1. The nucleic acids (ODN 2395 or R848) are packaged into virus-like particles (VLPs).

2. The VLPs are loaded with polymers (PEI) to ensure uptake.

3.4. Incubation of the Dendritic Cells with Naive CD4+ T Cells

The antigen-loaded dendritic cells were incubated with the naive CD4+ T cells at a ratio of 1:120.

The secretion of cytokines IL-4, IL-6, IL-10, IL-12 and IFN-γ is monitored over time. Further, the formation of Th2 surface markers CCR2, 3 and 4 is examined.

3.5. Activation of Naive B Cells

Thus, the naive B cells were isolated from the PBMCs in a highly pure form. In order to determine the antigen-specific activation, the B cells with the ZEA are incubated together with TLR7/9 agonists (ODN 2395 or R848). The activation is supported by antigen-specific Th2-T helper cells.

3.6. Orchestration of the Individual Components

Antibody-Secreting Plasma Cells

1. Generation of 1×10⁶ dendritic cells for 5 days with 500 U/ml of IL-4 and 800 U/ml of GM-CSF.

2. Addition of 5 μg of ZEA to the immature dendritic cells.

3. Addition of CD4+ T cells to mature dendritic cells at a ratio of 120 to 1 to generate Th2-T helper cells.

4. For antigen-specific activation, the B cells were incubated with the ZEA. The activation is supported by the addition of cytokines IL-2, IL-4, IL-6, IL-10.

5. Admixing the activated B cells with the activated Th2-T helper cells.

6. Incubation for up to ten days, the antibody secretion being checked each day.

3.7. Generation of B Cell Clones

Isolation and Immortalization of B Cells

In order to recover the B cell clones, the activated B cells are isolated from the corresponding system, and hybridomas may be formed by fusion with a myeloma cell. Selection generates an immortal cell line, which can be cultured in vitro and produces antibodies of a single specificity and isotype (Köhler & Milstein 1975). For this purpose, the lymphocytes were isolated from the system using a B-Cell Isolation Kit (Miltenyi Biotec). Subsequently, the cells were fused with the heteromyeloma cell line CB.F7 (Grunow et al., 1988) in the presence of polyethylene glycol (PEG). The cell seeds were sown at low cell counts in HAT medium in 96-well plates. After about two weeks, the supernatants of the cultures were examined for antibody production (Perryman L E et al., 1990).

3.8. Characterization of the Antibodies

A. Purification of the Antibodies

About two weeks after the fusion, the supernatants of the cultures were examined for antibody production, and the positive cultures were biologically cloned and expanded. The secreted antibodies were purified from the supernatants by affinity chromatography (Amersham).

B. Neutralization Capacity

For neutralization studies, the inhibition of the binding of the mycotoxin to different target cells was determined by means of the purified antibodies. Thus, the antigen was incubated with different antibody concentrations (diluted in 2-log steps) at RT for 45 min. This mixture was subsequently added to 1.5×10⁵ target cells/ml and incubated at 37° C. for 3 days. The detection of the bound mycotoxin was then effected by Western blotting. A concentration (ng/ml) at which complete protection was found in the cell cultures was shown as the neutralizing antibody concentration. FIG. 4 shows detection of in vitro generated ZEA-specific antibody by ELISA.

### LIST OF REFERENCES

1. Borrebaeck: Strategy for the production of human monoclonal antibodies using in vitro activated B cells. J. Immunol. Meth. 123: 157-165 (1989)

2. Chin, Malmborg, Kristensson, Hinkula, Wahren and Borrebaeck: Mimicking the humoral immune response in vitro results in antigen-specific isotype switching supported by specific autologous T helper cells: generation of human HIV-1-neutralizing IgG monoclonal antibodies from naïve donors. Eur. J. Immunol. 25: 657-663 (1995)

3. Ettinger, Sims, Fairhurst, Robbins, daSilva, Spolski, Leonard and Lipsky: IL-21 induces differentiation of human naïve and memory B cells into antibody-secreting plasma cells. J. Immunol. 176: 7867-7879 (2005)

4. Heilmann: Wechselwirkungen von Immunzellen mit synthetischen und biomimetrischen Oberflächen. Dissertation, Mathematisch-Naturwissenschaftliche Fakultät Universität Potsdam (2006)

5. Kawai and Akira: Innate immune recognition of viral infection. Nat. Immunol. 7: 131-137 (2006)

6. Köhler G & Milstein C. Continuous cultures of fused cells secreting antibody of pre-defined specificity. Nature; 256: 495-7 (1975).

7. Konthur, Hust and Dübel: Perspectives for systematic in vitro antibody generation. Gene 364: 19-29 (2005)

8. Li, Sai, Berger, Chao, Davidson, Deshmukh, Drozdowski et al.: Human antibodies for immunotherapy development generated via a human B cell hybridoma technology. Proc. Natl. Acad. Sci. USA 103: 3557-3562 (2006)

9. Mascelli, Zhou, Sweet, Getsy, Davis, Graham and Abernethy: Molecular, Biologic and Pharmacokinetic properties of monoclonal antibodies: Impact of these parameters on early clinical development. J. Clin. Pharmacol. OnlineFirst March 22 (2007)

10. Mohamadzadeh, Berard, Essert, Chalouni, Pulendran, Davoust, Bridges, Palucka and Banchereau: Interleukin 15 skews monocyte differentiation into dendritic cells with features of Langerhans Cells. J. Exp. Med. 194: 1013-1019 (2001).

11. Nicolaides, Sass and Grasso: Monoclonal antibodies: A morphing landscape for therapeutics. Drug Develop. Res. 67: 781-789 (2006)

12. Peters, Xu, Ruppert, Ostermeier, Friedrichs and Giseler: Signals required for differentiating dendritic cells from human monocytes in-vitro. Advan. Exp. Med. Biol. 329: 275-280 (1993).

13. Ruprecht and Lanzavecchia: Toll-like receptor stimulation as a third signal required for activation of human naive B cells. Eur. J. Immunol. 36: 810-816 (2006).

14. Tanaka, Demeure, Rubio, Delespesse and Sarfati: Human monocyte-derived dendritic cells induce naïve T cell differentiation into T helper Cell type 2 (Th2) or Th1/Th2 effectors: Role of stimulator/Responder Ratio. J. Exp. Med. 192: 405-411 (2000)

15. Traggiai, Becker, Subbarao, Kolesnikova, Uematsu, Gismondo, Murphy, Rappuoli and Lanzavecchia: An efficient method to make human monoclonal antibodies from memory B cells: potent neutralization of SARS coronavirus. Nat Med 10: 871-875 (2004)

16. Zafiropoulos, Andersson, Krambovitis and Borrebaeck: Induction of antigen-specific isotype switching by in vitro immunization of human naïve B lymphocytes. J. Immunol. Meth. 200: 181-190 (1997)

17. Xu, Katakura, Yamashita, Fang, Tamura, Matsumoto et al.: IL-10 augments antibody production in in vitro immunized lymphocytes by inducing a Th2-type response and B cell maturation. Biosci. Biotechnol. Biochem. 68: 2279-2284 (2004)

1. An in vitro process for the preparation of antibodies of the IgG type, comprising the steps of:
i. challenging dendritic cells obtained from a donor with an antigen against which the antibodies to be prepared are directed;
ii. challenging CD4+ T cells obtained from the same donor with fragments of the antigen presented by the dendritic cells from step i., with the proviso that the dendritic tells are ones essentially not secreting interferon-γ (IFN-γ) and interleukin 12 (IL-12), to generate antigen-specific CD4+ Th2 cells;
iii. challenging with the antigen a B cell population obtained from the same or a genetically similar donor and including a sufficient proportion of naive B cells; and
iv. contacting the antigen-specific CD4+ Th2 cells according to ii. with at least a fraction of the B cell population from iii. to form an antigen-specific plasma cell;
v. immortalizing said antigen-specific plasma cell; and
vi. isolating the IgG antibodies formed by said antigen-specific plasma cell.

2. The process according to claim 1, wherein the dendritic cells are differentiated from peripheral blood monocytes (PBMC) obtained from the donor.

3. The process according to claim 1, wherein the antibodies are of the type immunoglobulin G (subtypes 1-4).

4. The process according to claim 1, wherein the donors are mammals including humans.

5. The process according to claim 1, wherein the antigen is proteins, polypeptides, haptens and immunogenic substances, such as DNA and RNA.

6. The process according to claim 1, wherein the antigen-specific plasma cell is immortalized by infection with Epstein-Barr virus (EBV) or by fusion with a myeloma cell.

7. The process according to claim 1, wherein the antigen-specific plasma cell is cultured in a cell culture or bioreactor, and the antibodies are recovered from a culture fluid.

8. The process according to claim 1, wherein a monoclonal cell line is established from an antigen-specific plasma cell.
